(19) United States
(12) Patent Application Publication
(10) Pub. No.: US 2016/0168044 A1
(43) Pub. Date: Jun. 16, 2016

(54) CONVERTING GLYCEROL TO PROPYLENE

(71) Applicant: PHILLIPS 66 COMPANY, Houston, TX (US)

(72) Inventors: Tie-Pan SHI, Bartlesville, OK (US); Jianhua YAO, Bartlesville, OK (US); Brian C. DUNN, BARTLESVILLE, OK (US); Edward L. SUGHRUE, Edmond, OK (US)

(73) Assignee: PHILLIPS 66 COMPANY, Houston, TX (US)

(21) Appl. No.: 14/958,198

(22) Filed: Dec. 3, 2015

Related U.S. Application Data
(60) Provisional application No. 62/090,906, filed on Dec. 12, 2014.

(51) Int. Cl. C07C 1/20; C10G 50/00

(52) U.S. Cl. CPC C07C 1/20; C10G 50/00

(57) ABSTRACT

Processes relating to a one-step conversion to directly produce propylene from glycerol with a hydrotreating catalyst under a constrained hydrogen/glycerol feed ratio.

### STATEMENT REGARDING FEDERALLY SPONSORED RESEARCH OR DEVELOPMENT

This application is a non-provisional application which claims the benefit of and priority to U.S. Provisional Application Ser. No. 62/090,906 filed Dec. 12, 2014, titled “Converting Glycerol to Propylene,” which is incorporated by reference in its entirety.

### FIELD OF THE INVENTION

The present invention relates to a process that converts glycerol to propylene with high efficiency.

### BACKGROUND OF THE INVENTION

Glycerol (glycerin) is a by-product from the trans-esterification of triglycerides to biodiesel. Every gallon of biodiesel produced generates about half a kilogram of glycerol. With the expansion of the commercial biodiesel production, a large quantity of glycerol is produced. In fact, it has been projected that by 2016, as much as 4 billion gallons of glycerol might be produced. Unfortunately, glycerol has thus far proven difficult to convert into useful chemicals or fuels.

Propylene is the second most important raw material in the petrochemical industry after ethylene. It is the starting compound for the production of a wide-variety of chemicals, including polypropylene-based plastics, which account for nearly two-thirds of worldwide demand. Polypropylene is used for the production of films, packaging, caps and closures as well as myriad other applications. In the year 2008, the worldwide sales of polypropylene reached a value of over 90 billion dollars (USD).

Accordingly, developing of methods that would allow efficient and inexpensive conversion of glycerol to useful compounds, such as propylene, would be a useful addition to the art.

### BRIEF SUMMARY OF THE DISCLOSURE

The inventive methods disclosed herein provide an efficient way to derive more valuable products from glycerol, a low-value and abundant by-product of the transesterification of triglycerides to produce biodiesel. In certain embodiments, glycerol is converted to propylene in the presence of a limiting molar ratio of hydrogen gas to glycerol feedstock. Optionally, the propylene is utilized to make a liquid transportation fuel or a fuel additive.

Certain embodiments of the process comprise contacting a feedstock mixture comprising glycerol and hydrogen with a catalyst at a temperature in a range from 175° C. to 550° C., wherein limiting the molar ratio of hydrogen to glycerol increases the molar percentage of the glycerol that is converted to propylene. Optionally, limiting the molar ratio of hydrogen to glycerol simultaneously decreases the molar percentage of the glycerol that is converted to propane. Limiting the molar ratio of hydrogen to glycerol is optionally less than or equal to 6:1, 5:1, 4:1, or even 3:1.

In certain embodiments, the contacting of a feedstock mixture comprising glycerol and hydrogen with a catalyst is conducted at a molar ratio of hydrogen to glycerol that is in a range from 5.5:1 to 0.1:1, inclusive, optionally a range from 5:1 to 1:1, inclusive, from 4:1 to 1:1, inclusive, or even from 3:1 to 1:1, inclusive. The contacting is generally performed at a temperature in a range from 175° C. to 550° C. and a pressure in a range from 0 psig (0 bar) to 2900 psig (200 bar), and optionally converts the propylene to a product that can be utilized as a liquid transportation fuel or transportation fuel additive.

### DETAILED DESCRIPTION

Various exemplary embodiments of the inventive processes and systems will now be described in more detail. Glycerol is a by-product from the biodiesel esterification process. Using conventional hydrotreating conditions, glycerol is converted to propane, a relatively low-value hydrocarbon that can be used as a fuel gas. Saturation of glycerol in this manner follows the reaction pathway:

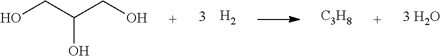

However, we have found that when the partial pressure of hydrogen decreased, hydrotreatment of glycerol can produce either propanol or propylene, according to stoichiometry:

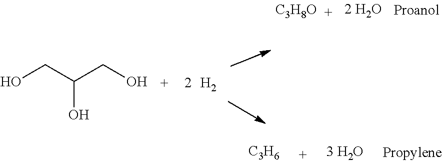

While not wishing to be bound by theory, the reaction pathway from glycerol to propylene is likely through the acrolein intermediate:

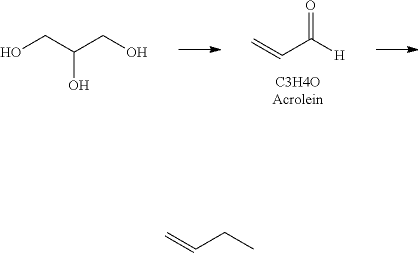

If so, this conversion from glycerol to propylene likely involves dehydration to remove two water molecules, followed by hydrogenation and a final dehydration of the acrolein aldehyde group to form propylene.

While it is known in the art that propylene can be formed from glycerol via an acrolein intermediate, the inventive processes described herein have the advantage of producing a yield of propylene from glycerol that is at least two orders of magnitude greater that previously shown, while utilizing comparable (if not lower) reaction temperatures, and with no detectable production of propane.

The feedstock generally comprises a glycerol stream. In certain embodiments, the feedstock may be a crude glycerol stream derived from biomass. Preferably, the glycerol stream is at least minimally filtered to remove any contaminants or solid particulates that may contaminate or inactivate the catalyst used for hydrotreating the feedstock. The feedstock may comprise water which optionally is separated prior to hydrotreating the feedstock.

The glycerol feedstock is mixed with hydrogen and contacted with a catalyst in a reaction zone that is suitable for converting the glycerol feedstock to propylene. In various embodiments the contacting occurs at a temperature in a range from 175° C. to 550° C., optionally 200° C. to 500° C., 225° C. to 450° C., 225° C. to 400° C., or from 200° C. to 300° C.

The pressure is generally maintained in a range from 200 psig to 1200 psig. The feedstock is generally hydrotreated for a period of time ranging from 0.1 to 2.5 hours. In certain embodiments, the feedstock is hydrotreated for a period of time in a range from 0.6 to 2.5 hours, optionally, 0.6 to 1.5 hours, or even in a range from 0.5 to 1.0 hours.

The catalyst used may comprise any catalyst suitable for a hydrotreating process. These catalysts are generally based on metals from groups VIB and VIII of the Periodic Classification of the Elements, such as molybdenum (Mo), tungsten (W), nickel (Ni) and cobalt (Co). The most commonly used hydrotreating catalysts are formulated from cobalt-molybdenum (Co—Mo), nickel-molybdenum (Ni—Mo) and nickel-tungsten (Ni—W) systems on porous inorganic supports, such as aluminas, silicas or silicas/aluminas. These catalysts, manufactured industrially in very large tonnages, are supplied to the user in their oxide forms (for example, cobalt oxides-molybdenum oxide catalysts on alumina, symbolized by the abbreviation: Co—Mo/alumina) of hydrotreating catalyst. In certain embodiments, the hydrotreating catalyst comprises trimetallic base metal oxides, including (but not limited to) catalysts comprising Mo—W—Ni, including any of the Nebula™ brand hydrotreating catalysts (Abermarle Corporation, USA). In certain embodiments, the catalyst may comprise mixed Fe—Mo sulfides and Fe—W sulfides. A second (Co or Ni) promoter may be added to the Fe—Mo or Fe—W catalyst to increase the catalyst activity and/or selectivity.

In addition to the combinations of Group VIII and Group VIb transition metal sulfides, the catalyst may comprise any transition metal sulfides of the 1st, 2nd and 3rd row of the Periodic Table, including single sulfides of V, Ru, Rh, Nb, Re and Pd. Besides single sulfides, the catalyst may alternatively comprise specific combinations of transition metal sulfides such as V—Mo, Cu—Mo, Ni—Ru, Ni—Rh, Co—Re, Ni—Re and Ni—Nd.

In certain embodiments, the catalyst may comprise said metals or metal combinations in oxide form. Such oxides may be reduced to completely or partially metal or metal alloys in the reactor startup step or during the regular operation. These variants are equally successful in performing the inventive processes disclosed herein.

The catalyst may be either supported or unsupported. In certain embodiments, unsupported catalysts are preferred, as the population of the active sites is much higher in unsupported catalysts and the total absence of the metal—support interaction makes unsupported Co/Ni—Mo/W sulfides the ultimate (high intrinsic activity) Type 2 catalysts. Also several noble metals (in particular Ru, Rh, Os and Ir) have very high intrinsic activities in different hydrotreating reactions and may be utilized in the catalyst as well. An extensive characterization of such hydrotreating catalysts and structural or substituted variants is well established in the art and is not critical to successfully performing the inventive processes disclosed herein.

The following examples are provided to better explain one or more of the various embodiments, and are not intended to limit or define the full scope of the inventive processes.

### Example 1

Glycerol was hydrotreated in a fixed bed reactor with a conventional hydrotreating catalyst at 600° F. (316° C.), 1200 psig, at a liquid hourly space velocity (LHSV) of 0.4 h⁻¹, and with a feedstock comprising a molar ratio of hydrogen/glycerol of 6.8:1. The results shown in Table 1 represent the average of five runs, and the conversion of glycerol for all runs was greater than 99%. The product distribution is shown in Table 1.

TABLE 1
Glycerol Hydrotreating Product Selectivity
at a H₂/glycerol ratio of 6.8:1

| Product | Selectivity, C (mol %) | Std. Dev. (mol %) |
|---|---|---|
| C1-C2 | 8.2 | 1.3 |
| Propane | 75.7 | 1.4 |
| Propylene | 0 | 0 |
| C4+ | 10.3 | 1.7 |
| CO + CO₂ | 5.8 | 1.3 |

### Example 2

In a second experiment, glycerol was hydrotreated while restricting the H2/glycerol ratio to 2.3. Otherwise, the experiment utilized the same conventional hydrotreating catalyst and experimental conditions as utilized in Example 1, (i.e., 600° F. (316° C.), 1200 psig, LHSV of 0.4 h⁻¹). In five separate runs, the conversion of glycerol was greater than 99%. The average experimental product distribution is shown in Table 2. When compared to the product profile depicted in Table 1, it is clear that decreasing the hydrogen/glycerol ratio from 6.8:1 to 2.3:1 unexpectedly resulted in a dramatic shift in product selectivity from propane to propylene.

TABLE 2
Glycerol Hydrotreating Product Selectivity
at an H₂/glycerol ratio of 2.3:1

| Product | Selectivity, (C mol %) | Std. Dev. (C mol %) |
|---|---|---|
| C1-C2 | 14.2 | 0.4 |
| Propane | 0 | 0 |
| Propylene | 59.2 | 0.4 |
| C4+ | 12.8 | 0.7 |
| CO + CO2 | 13.8 | 0.7 |

### Example 3:

The hydrotreatment of glycerol at a H₂/glycerol ratio of 2.3:1 was also performed over a range of temperatures. Table 3 indicates the product selectivity (in mol %) that was obtained when hydrotreating was performed at a temperature of 550° F. (288° C.), 520° F. (271° C.) and 490° F. (254° C.). All other reaction conditions were the same as those used in Examples 1 and 2. In the experiments shown above, glycerol hydrotreating at 600° F. (315° C.) had resulted in greater than 99% glycerol conversion. Table 3 shows that decreasing the hydrotreating temperature to 550° F., 520° F. or even 490° F. resulted in incomplete conversion of glycerol of up to 14.1% (carbon mol %). However, Table 3 also shows that decreasing the hydrotreating temperature correlated with an increase in selectivity to propylene as product.

TABLE 3
Decreasing Temperature Increases Selectivity to Propylene
When Hydrotreating Glycerol at Low H₂/glycerol Ratio

Temperature

| | 550° F. | 520° F. | 490° F. |
|---|---|---|---|
| | (288° C.) | (271° C.) | (254° C.) |
| Runs | 3 | 3 | 1 |
| Conversion, (C mol %) | 90.9 | 88.7 | 85.9 |

Selectivity, (C mol %)

| | | | |
|---|---|---|---|
| C1-C2 | 8.4 | 6.8 | 3.3 |
| Propane | 0 | 0 | 0 |
| Propylene | 68.2 | 73.7 | 82.1 |
| C4+ | 14.2 | 13.1 | 11.5 |
| CO + CO2 | 9.2 | 6.3 | 3.1 |

This is important, as any un-converted glycerol could easily be recycled to process in a commercial setting, and a relatively low hydrotreating temperature of 490° F. (254° C.) produced over 82% propylene with no detectable production of propane. The lower temperature was also beneficial in that considerably less C1-C2, CO and CO2 were produced than at higher temperatures.

### Example 4

Ethylene glycol was hydrotreated utilizing the same catalyst and conditions described in Example 1, except the ratio of hydrogen to ethylene glycol was varied from a high of 3.4:1 to a low of 1.2:1. Also, in once run, the LHSV of the glycerol feedstock was increased to 1.6 h⁻¹. In all tests, the conversion of ethylene glycol was greater than 99%, but no detectable ethylene was produced, including tests that limited the H2/ethylene glycol feed ratio.

TABLE 4
Product Selectivity from Hydrotreating of Ethylene Glycol.

| H2/ethylene glycol ratio | 3.4 | 1.7 | 1.2 | 2.2 |
|---|---|---|---|---|
| LHSV, h⁻¹ | 0.4 | 0.4 | 0.4 | 1.6 |
| Conversion (C mol %) | 99.9 | 99.9 | 99.9 | 99.8 |

Selectivity (C mol %)

| | | | | |
|---|---|---|---|---|
| Methane | 4.2 | 6.4 | 8.6 | 6.2 |
| Ethane | 76.5 | 62.9 | 50.1 | 56.4 |
| Ethylene | 0 | 0 | 0 | 0 |
| C3+ | 8.0 | 10.8 | 13.9 | 7.1 |
| CO + CO2 | 11.3 | 19.9 | 27.4 | 30.3 |

Although the systems and processes described herein have been described in detail, it should be understood that various changes, substitutions, and alterations can be made without departing from the spirit and scope of the invention as defined by the following claims. Those skilled in the art may be able to study the preferred embodiments and identify other ways to practice the invention that are not exactly as described herein. It is the intent of the inventors that variations and equivalents of the invention are within the scope of the claims, while the description, abstract and drawings are not to be used to limit the scope of the invention. The invention is specifically intended to be as broad as the claims below and their equivalents.

We claim:

1. A process for converting glycerol to propylene, comprising contacting a feedstock mixture comprising glycerol and hydrogen with a hydrotreating catalyst at a temperature in a range from 175° C. to 550° C., wherein limiting the molar ratio of hydrogen to glycerol increases the molar percentage of the glycerol that is converted to propylene.

2. The process according to claim 1, wherein the limiting additionally decreases the molar percentage of the glycerol that is converted to propane.

3. The process according to claim 1, wherein the hydrotreating catalyst comprises Ni and Mo and W.

4. The process according to claim 1, wherein the molar ratio of hydrogen to glycerol is less than or equal to 6:1.

5. The process according to claim 1, wherein the molar ratio of hydrogen to glycerol is less than or equal to 5:1.

6. The process according to claim 1, wherein the molar ratio of hydrogen to glycerol is less than or equal to 4:1.

7. The process according to claim 1, wherein the molar ratio of hydrogen to glycerol is less than or equal to 3:1.

8. The process according to claim 1, wherein the molar ratio of hydrogen to glycerol is in a range from 5.5:1 to 0.1:1, inclusive.

9. The process according to claim 1, wherein the molar ratio of hydrogen to glycerol is in a range from 5:1 to 1:1, inclusive.

10. The process according to claim 1, wherein the molar ratio of hydrogen to glycerol is in a range from 4:1 to 1:1, inclusive.

11. The process according to claim 1, wherein the molar ratio of hydrogen to glycerol is in a range from 3:1 to 1:1, inclusive.

12. The process according to claim 1, wherein the contacting is performed at a pressure in a range from 0 psig (0 bar) to 2900 psig (200 bar).

13. The process according to claim 1, wherein the contacting is performed at a temperature in a range from 175° C. to 550° C.

14. The process according to claim 1, wherein the contacting is performed at a temperature in a range from 200° C. to 500° C.

15. The process according to claim 1, wherein the contacting is performed at a temperature in a range from 225° C. to 450° C.

16. The process according to claim 1, wherein the contacting is performed at a temperature in a range from 225° C. to 400° C.

17. The process according to claim 1, wherein the contacting is performed at a temperature in a range from 200° C. to 300° C.

18. The process according to claim 1, additionally comprising converting the propylene to a liquid transportation fuel or a liquid transportation fuel additive.
